(19) United States
(12) Patent Application Publication
(10) Pub. No.: US 2024/0228552 A9
(43) Pub. Date: Jul. 11, 2024

(54) NANOPORE TWEEZER APPROACH FOR PROTEIN KINASE ALLOSTERIC DRUG SCREENING

(71) Applicant: UNIVERSITY OF MASSACHUSETTS, Boston, MA (US)

(72) Inventors: Min Chen, Boston, MA (US); Fanjun Li, Boston, MA (US)

(21) Appl. No.: 18/482,052

(22) Filed: Oct. 6, 2023

Related U.S. Application Data
(60) Provisional application No. 63/378,686, filed on Oct. 7, 2022.

(51) Int. Cl. C07K 14/245; B82Y 5/00; C12N 9/12; C12Q 1/48

(52) U.S. Cl. CPC C07K 14/245; B82Y 5/00; C12N 9/12; C12Q 1/485; C12Y 207/10001

(57) ABSTRACT

Disclosed herein are nanopore tweezer systems that can be used to screen for allosteric inhibitors of protein kinases. In some embodiments, the protein kinase is a mutant kinase that confers resistance to chemotherapeutic drugs during cancer treatment. Therefore, the disclosed systems and methods can be used to identify drugs for treating drug resistant cancers.

### CROSS-REFERENCE TO RELATED APPLICATIONS

This application claims benefit of U.S. Provisional Application No. 63/378,686, filed Oct. 7, 2022, which is hereby incorporated herein by reference in its entirety.

### STATEMENT REGARDING FEDERALLY SPONSORED RESEARCH OR DEVELOPMENT

This invention was made with Government Support under Grant Nos. GM115442 and A1156187 awarded by the National Institutes of Health. The Government has certain rights in the invention.

### SEQUENCE LISTING

This application contains a sequence listing filed in ST.26 format entitled “921301-1080 Sequence Listing” created on Oct. 4, 2023, having 12,264 bytes. The content of the sequence listing is incorporated herein in its entirety.

### BACKGROUND

Protein kinases are enzymes that catalyze the phosphorylation of substrate proteins, regulating a wide range of cellular pathways. Aberrant kinase activities are implicated in various human diseases, making the kinases essential drug targets (Cohen, P. Nat Rev Drug Discov 2002 1:309-315). Small-molecule kinase inhibitors have revolutionized the treatment of various diseases, especially cancers (Druker, B. J. et al. N Engl J Med 2001 344:1031-1037; Russo, A. et al. Oncotarget 2015 6:26814-26825). To date, over 70 kinase inhibitors have been approved for clinical use and approximately 80% are active site inhibitors targeting the highly conserved ATP binding site (Laufer, S. & Bajorath, J. J Med Chem 2022 65:891-892). However, the high structural and sequence similarities of the ATP site among the kinome lead to low selectivity of the ATP site inhibitors. Besides, the emerging drug-resistance caused by point mutations in the ATP site and remote from the ATP site has become a major problem (Bhullar, K. S. et al. Mol Cancer 2018 17:48).

The development of inhibitors targeting allosteric sites which are less conserved among the kinome, represents an appealing approach for kinase inhibition but challenging due to limited screening methods are available and the difficulties in identifying allosteric sites. At present, only four allosteric inhibitors (trametinib, cobimetinib, binimetinib and selumetinib) for MEK1/2 and one (asciminib) for BCR-ABL1 have been approved for clinical use (Pan, Y. & Mader, M. M. J Med Chem 2022 65:5288-5299). The allosteric sites are usually without clear geometric or chemical features, associated with less populated high energy state and are poorly accessible to current experimental methods (Carofiglio, F. et al. Molecules 2020 25). Computational approaches were used to assist allosteric sites discovery (Greener, J. G. & Sternberg, M. J. Curr Opin Struct Biol 2018 50:1-8; Kakarala, K. K. & Jamil, K. J Biomol Struct Dyn, 2021 1-22; Mingione, V. R. et al. J Mol Biol, 2022 167628). Traditional high-throughput screening approaches with biochemical and cellular assays, and relatively lower throughput biophysical methods such as surface plasmon resonance (SPR) and affinity selection mass spectroscopy (ASMS) are popular in hit generation, but whether the hits interact with the drug targets in an allosteric site needs to be further validated (Pan, Y. & Mader, M. M. J Med Chem 2022 65:5288-5299). Previously, a time-resolved fluorescence resonance energy transfer (TR-FRET) assay was developed for kinase allosteric inhibitor screening but required fluorophore-labeled tracer and antibody, which was costly (Lebakken, C. S. et al. J Biomol Screen 2009 14:924-935). Some other screening methods for allosteric inhibitors were developed based on the identified allosteric sites. For example, a well-characterized kinase allosteric site is the myristate site of Abl tyrosine kinase, which is a hydrophobic pocket on the C-lobe of Abl kinase domain (FIG. 1A). Binding of myristoylated N-terminal of the Abl kinase to the myristate site induces bending of its C-terminal helix al, stabilizing an autoinhibited conformation of the Abl (Nagar, B. et al. Cell 112, 2003 859-871). An NMR-based conformational assay (Jahnke, W. et al. J Am Chem Soc 2010 132:7043-7048) and a fluorescence-based FliK assay (Schneider, R. et al. J Am Chem Soc 2012 134:9138-9141) were developed to monitor the bending of the helix αl and specifically screen for myristate site inhibitors for the Abl kinase. The FliK assay was also used to monitor a specific conformational change around the allosteric site of Src kinase by labeling the fluorophore on a different position (Simard, J. R. et al. Nat Chem Biol 2009 5:394-396). However, the NMR and FliK assays cannot be easily adopted to other kinases and drug targets due to the challenges in identifying allosteric sites. Therefore, to assist the drug discovery, there is a need for a more generalizable approach for allosteric hit finding, requiring minimal knowledge on the allosteric sites and less modification or labeling of the drug targets.

### SUMMARY

Disclosed herein is a nanopore tweezer system that involves (i) a fluid-filled compartment separated by a membrane into a first chamber and a second chamber, wherein the fluid is an ionic solution; (ii) a CIyA nanopore disposed in the membrane with a cis pore lumen of about 7 nm and a trans pore lumen of about 3 nm; (iii) a protein kinase in the lumen of the CIyA nanopore; and (iv) electrodes configured for generating an electrical potential difference across the membrane to facilitate ionic flow through the CIyA nanopore from the first chamber to the second chamber, wherein the protein kinase has an active site that is blocked with an inhibitor.

In some embodiments, the protein kinase has a molecular weight in the range of 15-70 kDa. In some embodiments, the protein kinase is tyrosine kinase, and wherein the inhibitor is a tyrosine kinase inhibitor. For example, in some embodiments, the protein kinase is selected from the group consisting of Abl1, ACK, ALK, ARG (Abl2), Axl, BRK, BTK, CTK/MATK, EGFR, Eph family, EphA1, EphA2, EphA3 (HEK), EphB2, EphB4 (HTK), FAK (PTK2), FER, FES, FGFR1, FGFR2, FGFR3, FGFR4, FGR, FLT1 (VEGFR1), FLT3, FLT4 (VEGFR3), FMS (CSF1R), Fyn, HER2 (ErbB2), HER3 (ErbB3), HER4 (ErbB4), IGF1R, INSR, ITK, Jak1, Jak2, Jak3, KDR (FLK1, VEGFR2), Kit, LCK, LTK, LYN, Mer, Met, MusK, PDGFRα, PDGFRβ, PYK2, Ret, RON, ROR2, ROS, SRC, Syk, Tie2 (TEK), TrkA (NTRK1), TrkB (NTRK2), TrkC (NTRK3), Tyk2, TYRO3 (SKY), Yes, and Zap-70, or a mutant variant thereof.

In some embodiments, the inhibitor is an ATP-competitive inhibitor. For example, in some embodiments, the inhibitor is selected from the group consisting of dasatinib. imatinib, nilotinib, AIM-100, KRCA-0008, dorsomorphin, A-443654, TAE684, DMH-1, ML347, AZ12601011, AZD0156, KU-60019, Elimusertib, Gartisertib, VE-821, AK-01, Cabozantinib, βARK1 Inhibitor, Dorsomorphin, AZ304, DP-4978, LXH254, B12536, SF2523, Cpd 4f, XMU-MP-3, 2OH-BNPP1, BAY-524, AT7519, BMS-265246, SU9516, AT7519, Dinaciclib, Prexasertib, Prexasertib dihydrochloride, Prexasertib dimesylate, IC261, Hematein, Tpl2 Kinase Inhibitor 1, TC-DAPK6, HS38, BAY-8400, MBM-55, NH125, Erlotinib Hydrochloride, Lapatinib, ALW-II-41-27, NVP-BHG712, AWL-II-38.3, JI-101, Tesevatinib, XMD8-92, AX-15836, BAY885, Conteltinib, Defactinib, E260, Herbimycin A, ON123300, ENMD-2076, Infigratinib, ENMD-2076, Saracatinib, Linifanib, Cediranib, Linifanib, GW2580, TG 100572 Hydrochloride, PP2, RGB-286638, Indirubin-3′-monoxime, Varlitinib, Epertinib hydrochloride, Sapitinib, PF-06260933, Protein kinase inhibitors 1 hydrochloride, AZD-3463, Resveratrol, PS-1145, IMD-0560, TBK1/IKKε-IN-2, TBK1/IKKε-IN-5, ILK-IN-3, Ceritinib, IRAK inhibitor 2, Takinib, PF-06426779, HS271, BMS-509744, AZD-1480, SAR-20347, Ritlecitinib, AS601245, WHI-P258, Tyrphostin AG1433, Sorafenib, GA-017, S116836, SM1-71, Pim1/AKK1-IN-1, LRRK2-IN-1, GSK2578215A, R406, MKI-1, Trametinib, JNJ-47117096 hydrochloride, MELK-8a hydrochloride, UNC569, LDC1267, Tivantinib, Gossypetin, HRX-0215, Hesperadin, Rapamycin, AZ-23, ZINC05007751, JH 295, rac-CCT 250863, Talmapimod, RWJ-67657, LY-2584702 tosylate salt, FRAX597, FRAX486, GNE 2861, FRAX1036, Seralutinib, ISRIB (trans-isomer), GSK2656157, TCS PIM-1 1, SGI-1776, SEL24-B489, SGI-1776, SEL24-B489, 5-lodotubercidin, Staurosporine, Ruboxistaurin, Enzastaurin, Staurosporine, Mallotoxin, Spheciosterol sulfate A, Staurosporine, Sotrastaurin, PKR-IN-C16, PKR-IN-C51, Wortmannin, Conteltinib, TAK-632, ON123300, RKI 1447 dihydrochloride, AMG-208, BMS-777607, Lorlatinib, BRD7389, BIX 02565, BI-D1870, GSK 650394, PP121, Syk Inhibitor II, Piceatannol, Midostaurin, EW-7195, LY3200882, GW788388, S116836, Cabozantinib, Altiratinib, Entrectinib, Gandotinib, BMS-777607, WNK463, SU6656, PD173955, and RDN009 In some embodiments, the membrane preparation comprises a planar lipid bilayer. For example, the membrane preparation can be a micelle, a bacterium, or a eukaryotic cell.

In some embodiments, the protein kinase has been modified with an N-terminal positively charged peptide tag to extend the kinase residence time. For example, in some embodiments, the N-terminal positively charged peptide tag comprises the amino acid sequence KRSGGKK (SEQ ID NO:10), KRKKSKGG (SEQ ID NO:11), or KRKKSGG (SEQ ID NO:12).

In some embodiments, the CIyA pore comprises 10 to 14 subunits, wherein each subunit of the CIyA nanopore has the amino acid sequence SEQ ID NO:1, or a variant thereof having at least 80% sequence identity to SEQ ID NO:1.

In some embodiments, each subunit of the CIyA nanopore has at least one C87S, C87A, C285S or C285S substitution. In some embodiments, each subunit of the CIyA nanopore has at least one L99, E103, F166, and K294 substitution. In some embodiments, each subunit of the CIyA nanopore has the amino acid sequence SEQ ID NO:2 or SEQ ID NO:3.

Also disclosed herein is method for screening for an allosteric binder to a protein kinase that involves (a) providing the nanopore tweezer system of any one of claims 1 to 4, (b) assessing a gating pattern of the nanopore, (c) adding a candidate agent to the first chamber and/or the second chamber; (d) assessing the gating pattern for a change after step (c); and (e) comparing gating patterns from step (b) to step (d), wherein a change in the gating pattern is an indication that the candidate agent binds the peptide kinase at an allosteric site.

In some embodiments, the protein kinase is a mutant kinase that confers resistance to chemotherapeutic drugs during cancer treatment. Therefore, the disclosed methods can be used to identify drugs for treating drug resistant cancers.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1C show Abl kinase domain structures and schematics of CIyA nanopore sensing. FIG. 1A shows alignment of inhibitor bound Abl kinase domain structures. PDB 2HYY, 2GQG and 3K5V are used to generate Abl:imatinib, Abl:dasatinib and Abl:GNF-2 structures, respectively, with PyMOL. Abl kinase domains are colored in grey. N-lobe and C-lobe are indicated by the circles. Imatinib, dasatinib and GNF-2 are shown as stick. Corresponding chemical structures of the inhibitors are also shown. ATP site and Myristate site region are indicated with dashed rounded rectangle shapes. FIG. 1B shows schematics of CIyA nanopore set up for Abl kinase inhibitor detection. A model of the cross section of CIyA nanopore was shown and inserted in a planar lipid bilayer, with an AbN kinase domain trapped inside. Cis (grounded) and trans side of the setup are labeled on the graph. c, Schematics of open pore current (I_{O}) and blocked pore current (I_{B}). I_{O} was detected after a single nanopore inserted to the bilayer, I_{B} was detected when an analyte molecule (here is the AbN kinase) entered the nanopore.

Relative residual current (Iᵣₑₛ%) was calculated with Iᵣₑₛ%=100%*I_{B}/I_{O}. The current traces were collected at −80 mV in 150 mM NaCl, 100 mM Tris-HCl pH 7.5 buffer, with 100 nM _{N4pos}Abl kinase added to the cis chamber, at 22° C.

FIGS. 2A to 2L show detection of Abl kinase inhibitors with CIyA nanopore. FIGS. 2A, 2C, 2E, 2G, 2I, and 2K are schematics of inhibitor conditions and current traces (long and zoom-in traces) for apo _{N4pos}Abl (FIG. 2A), _{N4pos}Abl with 0.1 mM GNF-2 (FIG. 2C), with 0.1 μM dasatinib (FIG. 2E), with 0.1 μM imatinib (FIG. 2G), with 0.1 μM dasatinib and 0.1 mM GNF-2 (FIG. 2I), with 0.1 μM imatinib and 0.1 μM dasatinib (FIG. 2K). FIGS. 2B and 2D are histograms of only S1 state for apo _{N4pos}Abl (FIG. 2B), _{N4pos}Abl with 0.1 mM GNF-2 (FIG. 2D). FIGS. 2F, 2H, 2J, and 2L show all-events histogram for _{N4pos}Abl with 0.1 μM dasatinib (FIG. 2F), with 0.1 μM imatinib (FIG. 2H), with 0.1 μM dasatinib and 0.1 mM GNF-2 (FIG. 2J), with 0.1 μM imatinib and 0.1 μM dasatinib (FIG. 2L). Curve indicates the Gaussian multipeak fitting of histograms. The current traces were collected at −80 mV in 150 mM NaCl, 100 mM Tris-HCl pH 7.5 buffer, with 100 nM _{N4pos}Abl kinase and inhibitors added to the cis chamber, at 22° C.

FIGS. 3A to 3D show imatinib interactions with AbN kinases. FIG. 3A contains representative current trace of _{N4pos}Abl with 0.1 μM imatinib. The imatinib dissociation event was zoomed in. _{N4pos}Abl:imatinib complex state was indicated with a blue band, S1 state of apo _{N4pos}Abl was indicated with a yellow band, and S2 state of apo _{N4pos}Abl was indicated with a brown band. Imatinib dissociation moment was indicated by a blue arrow. FIG. 3B shows structure of H396P (PDB: 2F4J) and a representative trace of apo _{N4pos}H396P trapped with ClyA-AS nanopore. Zoom in images of S1, S2 states were also shown. FIGS. 3C to 3D contains representative current traces of _{N4pos}H396P with 0.1 μM imatinib. Imatinib dissociation and association events were zoomed in. _{N4pos}H396P:imatinib complex state, S1 state of apo _{N4pos}Abl, and S2 state of apo _{N4pos}Abl were indicated with orange, yellow and brown bands, respectively. Imatinib dissociation and association moments were indicated by blue and orange arrows, respectively. The current traces were collected at −80 mV in 150 mM NaCl, 100 mM Tris-HCl pH 7.5 buffer, with 100 nM Abl kinase and imatinib added to the cis chamber, at 22° C.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an agent can be an oligomer of nucleic acids, amino acids, or carbohydrates including, but not limited to proteins, peptides, oligonucleotides, ribozymes, DNAzymes, glycoproteins, RNAi agents (e.g., siRNAs), lipoproteins, aptamers, and modifications and combinations thereof. In some embodiments, an active agent is a nucleic acid, e.g., miRNA or a derivative or variant thereof.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

### CIyA Nanopores

Cytolysin A from Salmonella typhi (CIyA) is a biological nanopore that allows the investigation of natively folded proteins. The CIyA structure is ideal for this task because proteins can be electrophoretically trapped between the wide cis entrance (5.5 nm, table 1) and the narrower trans exit (3.3 nm), and can therefore be sampled for several minutes. Ionic currents through CIyA are so sensitive to the vestibule environment that blockades of human and bovine thrombin can be easily distinguished.

In some embodiments, the cytolysin is a modified CIyA from Salmonella typhi (S. typhi) or Salmonella paratyphi (S. paratyphi). For example, modified CIyA nanopores are described in U.S. Pat. No. 10,976,311, which is incorporated by reference herein for the teaching of this nanopore.

In some embodiments, the modified CIyA pore comprises a plurality of subunits, wherein each subunit comprises a polypeptide represented by an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to

(SEQ ID NO: 1)
MTGIFAEQTVEVVKSAIETADGALDLYNKYLDQVIPWKTFDETIKELSR
FKQEYSQEASVLVGDIKVLLMDSQDKYFEATQTVYEWAGVVTQLLSAYI
QLFDGYNEKKASAQKDILIRILDDGVKKLNEAQKSLLTSSQSFNNASGK
LLALDSQLTNDFSEKSSYYQSQVDRIRKEAYAGAAAGIVAGPFGLIISY
SIAAGVVEGKLIPELNNRLKTVQNFFTSLSATVKQANKDIDAAKLKLAT
EIAAIGEIKTETETTRFYVDYDDLMLSLLKGAAKKMINTSNEYQQRHGR
KTLFEVPDVGSSYHHHHH.

Accordingly, one or more amino acids may be substituted, deleted, and/or added, as compared with SEQ ID NO:1. Modifications may after the pore lumen in order to after the size, binding properties, and/or structure of the pore. Modifications may also after the CIyA pore outside of the lumen.

In certain embodiments, each subunit comprises a polypeptide represented by an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:1, wherein exactly one Cys residue is substituted with Ser, Ala. For example, the Cys residue may be C87 and/or C285 in SEQ ID NO:1. Other amino acid residues may be substituted, for example, with amino acids that share similar properties such as structure, charge, hydrophobicity, or hydrophilicity. In certain embodiments, substituted residues are one or more of L99, E103, F166, and K294. For example, the substituted residues may be one or more of L99Q, E103G, F166Y, and K294R. An exemplary subunit may comprise substitutions L99Q, E103G, F166Y, K294R, and C285S. An exemplary modified CIyA pore comprising subunits in which exactly one Cys residue is substituted with Ser may be called “CIyA-CS.” Thus, for example, each subunit may comprise a polypeptide represented by an amino acid sequence of

(SEQ ID NO: 2)
MTGIFAEQTVEVVKSAIETADGALDLYNKYLDQVIPWKTFDETIKELSR
FKQEYSQEASVLVGDIKVLLMIDSQDKYFEATQTVYEWCGVVTQLLSAY
IQLFDGYNEKKASAQKDILIRILDDGVKKLNEAQKSLLTSSQSFNNASG
KLLALDSQLTNDFSEKSSYYQSQVDRIRKEAYAGAAAGIVAGPFGLIIS
YSIAAGVIEGKLIPELNNRLKTVQNFFTSLSATVKQANKDIDAAKLKLA
TEIAAIGEIKTETETTRFYVDYDDLMLSLLKGAAKKMINTSNEYQQRHG
RKTLFEVPDV
or
(SEQ ID NO: 3)
MTGIFAEQTVEVVKSAIETADGALDLYNKYLDQVIPWKTFDETIKELSR
FKQEYSQEASVLVGDIKVLLMDSQDKYFEATQTVYEWAGVVTQLLSAYI
QLEDGYNEKKASAQKDILIRILDDGVKKLNEAQKSLLTSSQSFNNASGK
LLALDSQLTNDFSEKSSYYQSQVDRIRKEAYAGAAAGIVAGPFGLIISY
SIAAGVVEGKLIPELNNRLKTVQNFFTSLSATVKQANKDIDAAKLKLAT
EIAAIGEIKTETETTRFYVDYDDLMLSLLKGAAKKMINTSNEYQQRHGR
KTLFEVPDV

The modified CIyA pore may have a pore lumen of at least 3 nm in diameter, for example, the diameter may measure 3 nm, 3.5 nm, 4 nm, 4.5 nm, 5 nm, 5.5 nm, 6 nm, 6.5 nm, 7 nm, or greater. The size of the pore lumen may depend on the analyte to be detected by the modified CIyA pore. The cis diameter of the pore lumen may be at least 3.5 nm and/or the trans diameter of the CIyA pore may be at least 6 nm. In general, cis refers to the end of the modified CIyA pore to which an analyte is added, while trans refers to the end of the modified CIyA pore through which the analyte exits after translocating the length of the pore lumen. In artificial lipid bilayers, for example, the trans end of a pore may be inserted in the lipid bilayer, while the cis end of the pore remains on the same side of the lipid bilayer. Accordingly, the cis diameter of the pore is the diameter of the opening at the cis end of the pore, and the opening to which an analyte is added, while the trans diameter of the pore is the diameter at the opening of the trans end of the pore, from which an analyte exits.

The size of the pore lumen may also depend on the number of subunits in the modified CIyA pore. For example, larger pores which are made up of 13 or 14 subunits may have larger lumens than pores made up of 7 subunits. In some embodiments, the modified CIyA pores comprise 12 or more subunits. In certain embodiments, the modified CIyA pores comprise 12 subunits. In certain embodiments, the modified CIyA pores comprise 13 subunits, or comprise 14 subunits. The subunits may preferentially assemble in 12 mers and/or tamers, depending on the amino acid sequence of the subunits. In some embodiments, each subunit comprises a polypeptide as disclosed herein. Within a single modified CIyA pore, each of the subunits may be identical, or the subunits may be different, so that subunits in a modified CIyA pore may comprise sequences that differ from sequences of other polypeptide subunits in the same modified CIyA pore. In certain embodiments, modified CIyA pores as disclosed herein, such as CIyA-CS pores, may form more than one subtype depending on subunit composition. For example, there may be at least 2 or 3 different subtypes (i.e., Type I, Type II, Type Ill) of modified CIyA-CS pore, depending on subunits. Each subtype may have different conductance measurements, as compared with other subtypes. Subtypes may be preferentially formed by subunits of a particular polypeptide sequence.

The substitutions in specific residues may confer new properties on the modified CIyA pores, as compared with wild-type CIyA pores found in nature. Voltage dependent opening and closing (gating) of the pore at specific voltages is one property. In planar lipid bilayers, for example, CIyA-SS spontaneously opens and closes at applied potentials that are greater than +60 mV or lower than −90 mV. In some embodiments, the modified CIyA pores as described herein remain open when the voltage across the pore (i.e., the voltage across the membrane which the modified CIyA pore is in) ranges from +90 mV to −150 mV. Accordingly, the modified CIyA pore may remain open when the voltage across the pore is held at +90, +85, +80, +75, +70, +65, +60, +55, +50, +45, +40, +35, +30, +25, +20, +15, +10, +5, 0, −5, −10, −15, −20, −25, −30, −35, −40, −45, −50, −60, −65, −70, −75, −80, −85, −90, −95, −100, −110, −115, −120, −125, −130, −135, −140, −145, −150 mV, and/or the voltage across the pore is adjusted between +90 mV and −150 mV (inclusive), or any subrange of voltages in between. In certain embodiments, the modified CIyA pores show low electrical noise as compared with the signal (i.e., the current block measured). Thus, the noise inherent in a modified CIyA pore is reduced when pores as described herein are used. An exemplary modified CIyA pore shows noise measurements of _1.5 pA rms_to 3 pA rms_under_−35 mV in 150 mM NaCl 15 mM Tris.HCl pH 7.5_conditions. Notably, it is possible to reduce noise by increasing the salt concentration and/or altering the length of time during which a current block is measured.

In some embodiments, the modified CIyA pores show solubility properties that differ from wild-type CIyA pores. For examples, monomers of the modified CIyA pores may be soluble in water, and/or in other solutions where surfactants such as SDS or DDM are not present. Stable oligomers are modified CIyA pores that are capable of withstanding applied potentials of +150 mV to −150 mV across membranes or lipid bilayers into which the modified CIyA pores are inserted.

### Protein Kinases

As key regulators of most cellular pathways, protein kinases are frequently associated with diseases, either as causative agents or as therapeutic intervention points. Table 1 provides a list of known protein kinases associated with disease. Table 2 identifies some known active site inhibitors that could be used with the protein kinases.

TABLE 1
Protein Kinases Associated with Disease

| | | | Molecular | |
|---|---|---|---|---|
| Name | Group | Disease Type | Basis | Active Site Inhibitor |
| Abl1 | TK | Cancer | Trans | Imatinib, Dasatinib, Nilotinib |
| ACK | TK | Cancer | Amp | AIM-100, KRCA-0008 |
| ACTR2B | TKL | Development | Mut | dorsomorphin |
| Akt1 | AGC | Cancer | Amp, | A-443654 |
| | | | OE, Act | |
| Akt2 | AGC | Cancer, Diabetes | Amp, | A-443654 |
| | | | OE, Mut | |
| ALK | TK | Cancer | Trans | TAE684 |
| ALK1 | TKL | Cardiovascular | Mut | DMH-1, ML347 |
| (ACVRL1) | | | | |
| ALK2 | TKL | Development | Mut | DMH-1, ML347 |
| (ACVR1) | | | | |
| ALK4 | TKL | Cancer | Mut, | AZ12601011 |
| (ACVR1B) | | | Splice | |
| ANKK1 | TKL | Behavior | SNP | Not available |
| (SgK288) | | | | |
| ANPα | RGC | Hypertension | SNP, | Not available |
| (NPR1) | | | Expr | |
| ANPβ | RGC | Development | Mut | Not available |
| (NPR2) | | | | |
| ARG | TK | Cancer | Trans, | Imatinib, Dasatinib |
| (Abl2) | | | Expr | |
| ATM | Atypical | Cancer, CNS | LOF | AZD0156, KU-60019 |
| ATR | Atypical | Cancer, | Mut, | Elimusertib, Gartisertib, VE-821 |
| | | Development, | Splice | |
| | | Virology | | |
| AurB | Other | Cancer | OE | AK-01 |
| Axl | TK | Cancer | OE | Cabozantinib |
| BARK1 | AGC | Cardiac | Expr, | βARK1 Inhibitor |
| (GRK2, | | | Model | |
| ADRBK1) | | | | |
| Bcr | Atypical | Cancer | Trans | Not available |
| BMPR1A | TKL | Cancer | LOF | Dorsomorphin |
| (ALK3) | | | Mut | |
| BMPR1B | TKL | Development | Mut | Dorsomorphin |
| (ALK6) | | | | |
| BMPR2 | TKL | Cardiopulmonary | LOF | Not available |
| | | | Mut | |
| BRAF | TKL | Cancer, Cardiac, | GOF | AZ304, DP-4978, LXH254 |
| | | Development | Mut, | |
| | | | LOF | |
| | | | Mut | |
| BRD4 | Atypical | Cancer, Virology | Trans | BI2536, SF2523 |
| BRK | TK | Cancer | OE | Cpd 4f |
| BTK | TK | Cancer, Immunity | LOF | XMU-MP-3 |
| | | | Mut | |
| BUB1 | Other | Cancer | LOF | 2OH-BNPP1, BAY-524 |
| | | | Mut | |
| BUBR1 | Other | Cancer | Mut, | Not available |
| (BUB1B) | | | OE | |
| CAMK2δ | CAMK | Cardiovascular | Expr, | Not available |
| | | | Models, | |
| | | | Inh | |
| CDC2 | CMGC | Cancer | Act, | AT7519, BMS-265246 |
| (CDK1) | | | Splice | |
| CDK2 | CMGC | Cancer | | SU9516, BMS-265246 |
| CDK4 | CMGC | Cancer | Act, | AT7519, SU9516 |
| | | | GOF | |
| | | | Mut, | |
| | | | Amp, | |
| | | | Meth | |
| CDK5 | CMGC | Neurodegeneration | SNP | AT7519, Dinaciclib |
| CDK6 | CMGC | Cancer | OE, | AT7519 |
| | | | Trans | |
| CDK9 | CMGC | Cardiovascular, Viral | Expr | AT7519, Dinaciclib |
| | | infection | | |
| CDKL5 | CMGC | CNS, Development | Mut, | Not available |
| | | | Trans | |
| CHK1 | CAMK | Cancer | Mut | Prexasertib |
| (CHEK1) | | | | |
| CHK2 | CAMK | Cancer | Mut | Prexasertib dihydrochloride, |
| | | | | Prexasertib dimesylate |
| CK1δ, | CK1 | Neurodegeneration | | IC261 |
| CK1α | | | | |
| CK1ε | CK1 | Behavior, Cancer | SNP, | IC261 |
| (CSNK1ε) | | | Mut, | |
| | | | LOH | |
| CK2α1, | Other | Cancer, Circadian | OE, Act | Hematein |
| CK2α2 | | Rhythm, | | |
| (CSNK2α1/2) | | Neurodegeneration | | |
| COT/TPL2 | STE | Cancer, Inflammation | OE, | Tpl2 Kinase Inhibitor 1 |
| | | | Amp, | |
| | | | Mut | |
| CTK/MATK | TK | Cancer | OE | Not available |
| CYGD | RGC | Vision | Mut | Not available |
| (GUCY2D) | | | | |
| CYGF | RGC | Cancer | Mut | Not available |
| (GUCY2F) | | | | |
| DAPK1 | CAMK | Cancer, Epilepsy | Meth, | TC-DAPK 6, HS38 |
| | | | Expr | |
| DCAMKL1 | CAMK | Neurodegeneration | | Not available |
| DMPK1 | AGC | Neurodegeneration | Mut | Not available |
| DNAPK | Atypical | Cancer | Mut | BAY-8400 |
| DYRK1A | CMGC | Cognition | | MBM-55 |
| eEF2K | Atypical | Cancer | OE, Act | NH125 |
| (CaMKIII) | | | | |
| EGFR | TK | Cancer | Amp, | Erlotinib Hydrochloride, Lapatinib |
| | | | OE, | |
| | | | GOF | |
| | | | Mut | |
| Eph | TK | Cancer, Sensory | | ALW-II-41-27 |
| family | | | | |
| EphA1 | TK | Cancer | Expr | Not available |
| EphA2 | TK | Cancer | OE | NVP-BHG712 |
| EphA3 | TK | Cancer | Mut | AWL-II-38.3 |
| (HEK) | | | | |
| EphB2 | TK | Cancer | OE, | Not available |
| | | | Mut | |
| EphB4 | TK | Cancer | OE | JI-101, Tesevatinib |
| (HTK) | | | | |
| Erk5 | CMGC | Cancer, | Expr | XMD8-92, AX-15836, BAY885 |
| (BMK1) | | Cardiovascular | | |
| FAK | TK | Cancer | OE, | Conteltinib, Defactinib |
| (PTK2) | | | Amp, | |
| | | | Act | |
| FER | TK | Cancer | Expr | E260 |
| FES | TK | Cancer | Mut, | Herbimycin A |
| | | | Trans? | |
| FGFR1 | TK | Cancer, | Mut, | ON123300, ENMD-2076, Infigratinib |
| | | Development | Trans | |
| FGFR2 | TK | Cancer, | Mut, | ENMD-2076, Infigratinib |
| | | Development | Amp | |
| FGFR3 | TK | Cancer, | GOF | Infigratinib |
| | | Development | Mut, | |
| | | | Trans | |
| FGFR4 | TK | Cancer | SNP | Infigratinib |
| FGR | TK | Cancer | Amp | Saracatinib |
| FLT1 | TK | Cancer | Meth, | Linifanib, Cediranib |
| (VEGFR1) | | | OE | |
| FLT3 | TK | Cancer | GOF | Linifanib |
| | | | Mut | |
| FLT4 | TK | Lymphangiogenesis | Act, | Cediranib |
| (VEGFR3) | | | LOF | |
| | | | Mut | |
| FMS | TK | Cancer | Mut | GW2580 |
| (CSF1R) | | | | |
| Fyn | TK | Cancer, Epilepsy | | TG 100572 Hydrochloride, PP2 |
| GRK4 | AGC | Hypertension | SNP | Not available |
| (GPRK4) | | | | |
| GSK3 (α | CMGC | Cardiovascular, | SNP, | RGB-286638, Indirubin-3′-monoxime |
| & β) | | CNS, Diabetes, | Act | |
| | | Neurodegeneration | | |
| HER2 | TK | Cancer | Amp, | Varlitinib, Epertinib hydrochloride |
| (ErbB2) | | | OE | |
| HER3 | TK | Cancer | OE | Sapitinib |
| (ErbB3) | | | | |
| HER4 | TK | Cancer | Expr | Varlitinib, Epertinib hydrochloride |
| (ErbB4) | | | | |
| HGK | STE | Cancer | OE | PF-06260933 |
| (ZC1) | | | | |
| HIPK1 | CMGC | Cancer | OE | Protein kinase inhibitors 1 hydrochloride |
| HIPK2 | CMGC | Cancer | LOH, | Protein kinase inhibitors 1 hydrochloride |
| | | | Expr | |
| IGF1R | TK | Cancer, Growth, | Mut, | AZD-3463 |
| | | Longevity | SNP, | |
| | | | OE | |
| IKKα, | Other | Cancer, Diabetes, | | Resveratrol, PS-1145, IMD-0560 |
| IKKβ | | Inflammation | | |
| IKKε | Other | Cancer, Inflammation | Amp, | TBK1/IKKε-IN-2, TBK1/IKKε-IN-5 |
| | | | Expr, | |
| | | | Inh | |
| ILK | TKL | Cancer | OE | ILK-IN-3 |
| INSR | TK | Diabetes, | Mut, | Ceritinib |
| | | Development | SNP | |
| IRAK2 | TKL | Cancer, Inflammation | Mut | IRAK inhibitor 2 |
| IRAK4 | TKL | Infection | Mut | Takinib, PF-06426779, HS271 |
| ITK | TK | Cancer, Virology | Inh | BMS-509744 |
| | | | Trans | |
| Jak1 | TK | Cancer | Mut, | AZD-1480, SAR-20347 |
| | | | Act | |
| Jak2 | TK | Cancer | Trans | AZD-1480, SAR-20347 |
| Jak3 | TK | Cancer, Immunity | LOF | Ritlecitinib, SAR-20347 |
| | | | Mut | |
| JNK1 | CMGC | Cancer, Diabetes, | | AS601245 |
| | | Inflammation | | |
| JNK3 | CMGC | Cancer, CNS | Expr | WHI-P258 |
| KDR | TK | Cancer | Mut | Tyrphostin AG1433 |
| (FLK1, | | | | |
| VEGFR2) | | | | |
| Kit | TK | Cancer, | GOF | Sorafenib |
| | | Depigmentation | Mut, | |
| | | | LOF | |
| | | | Mut, | |
| | | | Act? | |
| LATS1 | AGC | Cancer | Meth, | GA-017 |
| | | | Expr, | |
| | | | Mut | |
| LATS2 | AGC | Cancer | Expr | GA-017 |
| LCK | TK | Cancer, Immunity | Trans, | S116836 |
| | | | Mut, | |
| | | | Expr, | |
| | | | Splice | |
| LIMK1 | TKL | Cancer, | OE, | SM1-71 |
| | | Development | Amp, | |
| | | | LOH | |
| LKB1 | CAMK | Cancer | LOF | Pim1/AKK1-IN-1 |
| STK11 | | | Mut | |
| LRRK2 | TKL | Cognition | Mut | LRRK2-IN-1, GSK2578215A |
| LTK | TK | Immunity | SNP | Not available |
| LYN | TK | Cancer | Act | R406 |
| MASTL | AGC | Hematopoiesis | Mut | MKI-1 |
| MEK1, 2 | STE | Cancer, Cardiac, | Mut, | Trametinib |
| (MAP2K1, 2) | | Development, | OE | |
| | | Virology | | |
| MELK | CAMK | Cancer | Expr, | JNJ-47117096 hydrochloride, MELK-8a |
| | | | Inh | hydrochloride |
| Mer | TK | Vision | LOF | UNC569, LDC1267 |
| | | | Mut | |
| Met | TK | Cancer | GOF | Tivantinib |
| | | | Mut, | |
| | | | OE, | |
| | | | Trans | |
| MISR2 | TKL | Reproduction | Mut | Not available |
| (AMHR2) | | | | |
| MKK3 | STE | Cancer | Mut, | Gossypetin |
| (MAP2K3) | | | Del | |
| MKK4 | STE | Cancer | LOF | HRX-0215 |
| (MAP2K4) | | | Mut, | |
| | | | Del | |
| MLK4 | TKL | Cancer | Mut | Not available |
| skMLCK | CAMK | Cardiovascular | Mut | Not available |
| (MYLK2) | | | | |
| smMLCK | CAMK | Hypertension | Model | Not available |
| (MYLK) | | | | |
| Mst4 | STE | Cancer | OE | Hesperadin |
| mTOR | Atypical | Cancer | | Rapamycin |
| (FRAP) | | | | |
| MusK | TK | Neuro | Mut, | AZ-23 |
| | | | Auto- | |
| | | | antibodies | |
| MYO3A | STE | Sensory | Mut | Not available |
| NEK1 | Other | Development, Renal | | ZINC05007751 |
| NEK2 | Other | Cancer | OE | JH 295, rac-CCT 250863 |
| NEK8 | Other | Cancer, Renal | OE | Not available |
| p38 | CMGC | Cancer, Inflammation | | Talmapimod, RWJ-67657 |
| α, β, γ, δ | | | | |
| p70S6K | AGC | Cancer, Diabetes | OE, | LY-2584702 tosylate salt |
| (RPS6KB1) | | | Amp | |
| PAK3 | STE | Cognition | LOF | FRAX597, FRAX486 |
| | | | Mut | |
| PAK4 | STE | Cancer | OE | GNE 2861, FRAX1036 |
| PDGFRα | TK | Cancer, | Trans, | Seralutinib |
| | | Development | Del, | |
| | | | Mut | |
| PDGFRβ | TK | Cancer | Trans, | Seralutinib |
| | | | OE | |
| PEK | Other | Diabetes | LOF | ISRIB (trans-isomer), GSK2656157 |
| (PERK) | | | Mut | |
| PHKγ2 | CAMK | Metabolism | LOF | Not available |
| | | | Mut | |
| Pim-1 | CAMK | Cancer | Trans, | TCS PIM-1 1, SGI-1776, SEL24-B489 |
| | | | OE, | |
| | | | Mut | |
| Pim-2 | CAMK | Cancer | | SGI-1776, SEL24-B489 |
| Pim-3 | CAMK | Cancer | | SGI-1776, SEL24-B489 |
| PINK1 | Other | Cancer, | Mut, | Not available |
| | | Neurodegeneration | Expr | |
| PKACα | AGC | Cancer | | Not available |
| PKC | AGC | CNS | Inh | 5-Iodotubercidin |
| (multi- | | | | |
| isoform) | | | | |
| PKCα | AGC | Cancer, | Mut, | Staurosporine |
| | | Cardiovascular | Del, | |
| | | | OE, Act | |
| PKCβ | AGC | Autism, Cancer, | SNP | Ruboxistaurin, Enzastaurin |
| | | Diabetes | | |
| PKCγ | AGC | Neurodegeneration, | Mut | Staurosporine |
| | | Pain | | |
| PKCδ | AGC | Cancer, | Expr | Mallotoxin |
| | | Cardiovascular, CNS | | |
| PKCε | AGC | Cancer, | Amp, | Spheciosterol sulfate A |
| | | Cardiovascular, CNS | Mut | |
| PKCη | AGC | Cancer | | Staurosporine |
| PKCθ | AGC | Cancer, Immunity | | Sotrastaurin |
| PKR | Other | Cancer, | Mut, | PKR-IN-C16, PKR-IN-C51 |
| (PRKR) | | Virology, | Expr, | |
| | | Neurodegeneration | Act | |
| PLK1 | Other | Cancer | Expr | Wortmannin |
| PRKX | AGC | Reproduction | Trans | Not available |
| PRKY | AGC | Reproduction | Trans | Not available |
| PYK2 | TK | Osteoporosis | Model, | Conteltinib |
| | | | Inh, | |
| | | | Expr | |
| Raf1 (c- | TKL | Cancer | Amp | TAK-632 |
| Raf) | | | | |
| Ret | TK | Cancer, | LOF | ON123300 |
| | | Development | and | |
| | | | GOF | |
| | | | Mut, | |
| | | | Trans | |
| RHOK | AGC | Vision | LOF | Not available |
| | | | Mut | |
| RNAseL | Other | Cancer, Virology | Mut | Not available |
| ROCK1, | AGC | Cardiovascular, | | RKI 1447 dihydrochloride |
| ROCK2 | | Hypertension, | | |
| | | Neurodegeneration | | |
| RON | TK | Cancer | OE, | AMG-208, BMS-777607 |
| | | | Splice | |
| ROR2 | TK | Development | Mut | Not available |
| ROS | TK | Cancer | Trans | Lorlatinib |
| RSK2 | AGC | CNS, Development, | LOF | BRD7389, BIX 02565, BI-D1870 |
| (RPS6KA3) | | Virology | Mut | |
| SGK1 | AGC | Cancer, | Expr | GSK 650394 |
| | | Cognition, Diabetes | | |
| SRC | TK | Cancer | Mut, | PP121, Dasatinib |
| | | | OE, Act | |
| Syk | TK | Allergy, Cancer | Meth, | Syk Inhibitor II, Piceatannol, Midostaurin |
| | | | Splice | |
| TGFβR1 | TKL | Cancer, | SNP, | EW-7195, LY3200882, GW788388 |
| (ALK5) | | Development | Mut | |
| TGFβR2 | TKL | Cancer, | LOF | GW788388 |
| | | Development, | Mut | |
| | | Fibrosis | | |
| Tie2 | TK | Angiogenesis, | Mut, | S116836, Cabozantinib, Altiratinib |
| (TEK) | | Cancer | OE | |
| TITIN | CAMK | Cardiovascular | Mut | Not available |
| TrkA | TK | Cancer, Sensory | Mut, | Entrectinib |
| (NTRK1) | | | Trans | |
| TrkB | TK | Cancer | Mut | Entrectinib |
| (NTRK2) | | | | |
| TrkC | TK | Cancer | Trans, | Entrectinib |
| (NTRK3) | | | Mut | |
| Tyk2 | TK | Immunity | SNP, | Gandotinib |
| | | | Model | |
| TYRO3 | TK | Cancer | OE | BMS-777607 |
| (SKY) | | | | |
| Wnk1 | Other | Hypertension | Intronic | WNK463 |
| | | | Mut | |
| Wnk4 | Other | Hypertension | Mut | WNK463 |
| Yes | TK | Cancer | Amp, | SU6656, PD173955 |
| | | | Act | |
| Zap-70 | TK | Immunity | Mut | RDN009 |

Molecular Basis Key:
Act—Activated
Amp—Amplified
Del—Deleted
Expr—Expression
GOF—Gain-of-function
Inh—Inhibitor Studies
LOF—Loss-of-function
LOH—Loss-of-heterozygosity
Meth—Methylation
Model—model organism studies
Mut—Mutation
OE—Overexpression
SNP—Single Nucleotide Polymorphism
Splice—Splicing change
Trans—Translocation

TABLE 2
Protein Kinase Inihbitors

| Name | Target | Class |
|---|---|---|
| Adavosertib | WEE1 | Small molecule |
| Afatinib | EGFR/ErbB2 | Small molecule |
| Axitinib | VEGFR1/VEGFR2/VEGFR3/ | Small molecule |
| | PDGFRB/c-KIT | |
| Bosutinib | Bcr-Abl/SRC | Small molecule |
| Cetuximab | EGFR | Monoclonal antibody |
| Cobimetinib | MEK | Small molecule |
| Crizotinib | ALK/Met | Small molecule |
| Cabozantinib | RET/MET/VEGFR2 | Small molecule |
| Dacomitinib | EGFR/ErbB2/ErbB4 | Small molecule |
| Dasatinib | multiple targets | Small molecule |
| Entrectinib | TrkA/TrkB/TrkC/ROS1/ALK | Small molecule |
| Erdafitinib | FGFR | Small molecule |
| Erlotinib | EGFR | Small molecule |
| Fostamatinib | Syk | Small molecule |
| Gefitinib | EGFR | Small molecule |
| Ibrutinib | BTK | Small molecule |
| Imatinib | Bcr-Abl | Small molecule |
| Lapatinib | EGFR/ErbB2 | Small molecule |
| Lenvatinib | VEGFR2 | Small molecule |
| Mubritinib | ? | Small molecule |
| Nilotinib | Bcr-Abl | Small molecule |
| Pazopanib | VEGFR2/PDGFR/c-kit | Small molecule |
| Pegaptanib | VEGF | RNA Aptamer |
| Ruxolitinib | JAK | Small molecule |
| Sorafenib | multiple targets | Small molecule |
| Sunitinib | multiple targets | Small molecule |
| SU6656 | Src, others | Small molecule |
| Tucatinib | HER2 | Small molecule |
| Vandetanib | RET/VEGFR/EGFR | Small molecule |
| Vemurafenib | BRAF | Small molecule |

### Membrane Preparation

Disclosed herein is a composition comprising one or more nanopores disposed in a membrane preparation. In certain embodiments, the membrane preparation is a synthetic membrane preparation. In some embodiments, the membrane preparation is a planar lipid bilayer. In certain embodiments, the membrane preparation is a micelle. In some embodiments, the membrane preparation is a membrane of a biological cell. In certain embodiments, the biological cell is a bacterium. In some embodiments, the biological cell is a eukaryotic cell.

In some embodiments, the nanopores are disposed in a membrane preparation such that on one side of the membrane preparation the nanopores are exposed to a common chamber and on the other side of the membrane preparation each nanopores is exposed to a separate chamber. In some embodiments, the extracellular loop of each nanopores is exposed to the common chamber. In some embodiments, the extracellular loop of each nanopores is exposed to the separate chamber. In some embodiments, the nanopores are configured for multi-nanopore high-throughput measurements. In some embodiments, a set of nanopores are provided in a membrane preparation (e.g., in a 2-dimensional array) such that at one side of the membrane preparation the multiple nanopores are exposed to a single chamber, and at the other side of the membrane preparation, each nanopore is exposed to an individual chamber.

### Screening Methods

Disclosed herein is a method of identifying allosteric kinase inhibitors that can be used to treat diseases associated with aberrant expression or activity of the kinase. The method can comprise providing the nanopore tweezer composition disclosed herein, assessing a gating pattern of the nanopore, exposing the cis side of the nanopore tweezer composition to a candidate agent; and assessing the gating pattern for a change, wherein a change in the gating pattern is an indication that the candidate agent binds the peptide kinase at an allosteric site.

In some embodiments, binding of a candidate agent to the protein kinase results in a conformational change in the nanopore that alters ion current flow through the pore. In some embodiments, the change in ion current flow is a result of a change in gating due to the conformational change.

In certain embodiments, determining the gating pattern comprises performing a single channel recording of ion flow through the nanopore. In some embodiments, the single channel recording is performed while exposing the nanopore to a potential in a range of −250 to +250 mV or −100 to +100 mV. In certain embodiments, the single channel recording is performed while exposing the nanopore to a potential in a range of −50 mV to +50 mV. In some embodiments, binding of the target to the heterologous peptide is detected based on an alteration (e.g., an increase or a reduction) in the gating pattern resulting from exposing the nanopore to a target molecule. In some embodiments, binding of the target to the heterologous peptide is detected based on an alteration (e.g., an increase or a reduction) in amplitude and/or frequency of gating. In some embodiments, alterations in gating patterns are due to the interaction of bound analyte with one or more loops at the ligand side of a beta barrel of a nanopore. In some embodiments, an analyte can slow down or otherwise alter the movement of a loop, e.g., by containing or tethering the loop, or altering the loop such that it is stuck in a “half-open” or “closed” position, etc.

In certain embodiments, the reduction is a reduction of the frequency and/or amplitude of gating events. In some embodiments, determining the gating pattern comprises determining a gating frequency (f, events/s). In certain embodiments, the gating frequency is a relationship between a total number of gating events and a recording time. In some embodiments, determining the gating pattern comprises determining a gating probability (Pgating). In certain embodiments, the gating probability is a relationship between a total time for which a pore is in a closed or partially closed state and a total recording time (total open and closed time). In some embodiments, determining the gating pattern comprises determining loop dynamics to detect ligand-target interactions. In some embodiments, a gating pattern change is from “quiet” to “noisy”. However, in some embodiments, a gating pattern change is from noisy to quiet. In some embodiments, a reduction is gating pattern from noisy to quiet occurs where a reduction in the ionic current noise is greater than 10%.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) used.

Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from purveyors of chemical libraries including but not limited to ChemBridge Corporation (16981 Via Tazon, Suite G, San Diego, CA, 92127, USA, www.chembridge.com); ChemDiv (6605 Nancy Ridge Drive, San Diego, CA 92121, USA); Life Chemicals (1103 Orange Center Road, Orange, CT 06477); Maybridge (Trevillett, Tintagel, Cornwall PL34 0HW, UK)

Alternatively, libraries of natural compounds in the form of bacterial, fungal, plant, and animal extracts are commercially available from a number of sources, including O2H, (Cambridge, UK), MerLion Pharmaceuticals Pte Ltd (Singapore Science Park II, Singapore 117528) and Galapagos NV (Generaal De Wittelaan L11 A3, B-2800 Mechelen, Belgium).

In addition, natural and synthetically produced libraries are produced, if desired, according to methods known in the art, e.g., by standard extraction and fractionation methods or by standard synthetic methods in combination with solid phase organic synthesis, micro-wave synthesis and other rapid throughput methods known in the art to be amenable to making large numbers of compounds for screening purposes. Furthermore, if desired, any library or compound, including sample format and dissolution is readily modified and adjusted using standard chemical, physical, or biochemical methods.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having allosteric inhibition activity. The same assays described herein for the detection of activities in mixtures of compounds can be used to purify the active component and to test derivatives thereof. Methods of fractionation and purification of such heterogeneous extracts are known in the art. If desired, compounds shown to be useful agents for treatment are chemically modified according to methods known in the art. Compounds identified as being of therapeutic value may be subsequently analyzed using in vitro cell based models and animal models for diseases or conditions, such as those disclosed herein.

Candidate agents encompass numerous chemical classes, but are most often organic molecules, e.g., small organic compounds having a molecular weight of more than 100 and less than about 2,500 Daltons. Candidate agents can include functional groups necessary for structural interaction with proteins, particularly hydrogen bonding, and typically include at least an amine, carbonyl, hydroxyl or carboxyl group, for example, at least two of the functional chemical groups. The candidate agents often contain cyclical carbon or heterocyclic structures and/or aromatic or polyaromatic structures substituted with one or more of the above functional groups.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Introduction

Nanopore technology has been used for a wide variety of applications, such as nucleic acids sequencing (Kasianowicz, J. J., et al. Proc Natl Acad Sci USA 1996 93:13770-13773; Manrao, E. A. et al. Nat Biotechnol 2012 30:349-353), proteomics and peptide sequencing (Brinkerhoff, H., et al. Science 2021 374:1509-1513), single-molecule chemistry (Qing, Y. et al. Angew Chem Int Ed Engl 2020 59:15711-15716; Pulcu, G. S., et al. Nat Nanotechnol 2015 10:76-83), single-molecule enzymology (Galenkamp, N. S. & Maglia, G. ACS Catal 2022 12:1228-1236), and the detection of various molecules including metal ions (Heaton, I. & Platt, M. Industrial & Engineering Chemistry Research 2020 59:21403-21412), antibodies (Fahie, M., et al. ACS Nano 2015 9:1089-1098; Fahie, M. A., et al. Analytical chemistry 2015 87:11143-11149) and enzymes (Harrington, L., et al. Proc Natl Acad Sci USA 2013 110:E4417-4426; Pham, B. et al. Biophysical journal 2019 117:844-855; Meng, F. N., et al. Anal Chem 2019 91:9910-9915; Shorkey, S. A., et al. Chembiochem 2021 22:2688-2692), due to the advantages of fast detection speed, high selectivity and sensitivity, label-free and real-time monitoring. Moreover, nanopore devices can operate in high throughput with multiplexing capabilities at low-cost (Wang, M. et al. Small 2020 16:e2002169; Cardozo, N. et al. Nat Biotechnol 2022 40:42-46). Typically, a nanopore sensor consists of a nanosized aperture in a voltage-biased membrane which separates two chambers containing electrolyte solution. The identities of analytes are probed from the current blockages induced by the analytes interacting with the nanopore. More recently, Cytolysin A (ClyA) nanopores were used to study protein-ligand interactions by trapping ligand-binding proteins inside the nanopore, the specific interactions between the ligands and the trapped proteins were visualized through the current changes (Galenkamp, N. S., et al. Nat Commun 2018 9:4085; Li, X., et al. ACS Nano 2020 14:1727-1737; Li, F., et al. Nat Commun 2022 13:3541).

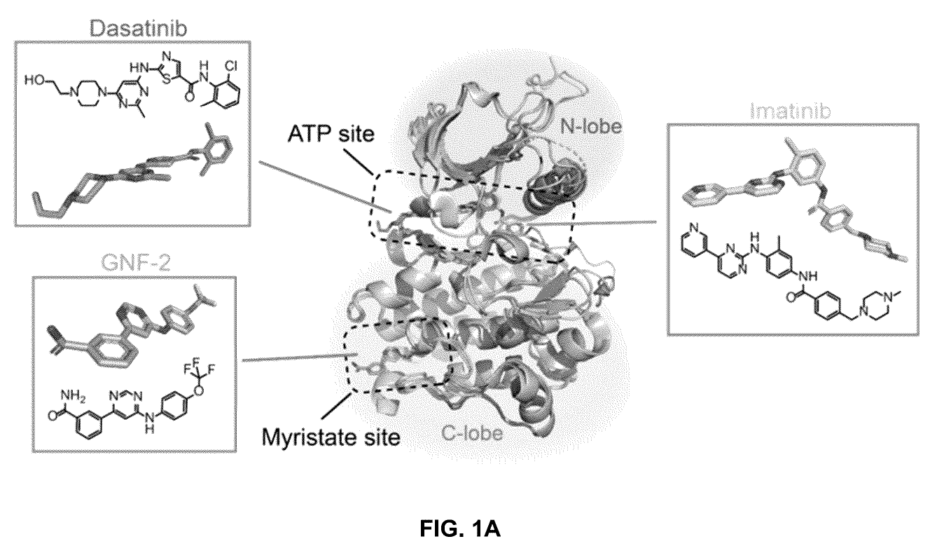

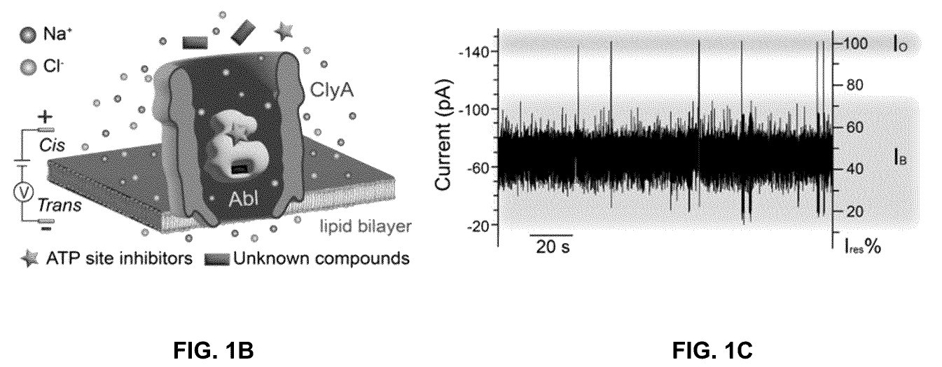

Disclosed herein is a generalizable label-free nanopore-based approach for detecting allosteric kinase binders by monitoring the ionic current signatures induced by different compounds binding to the kinase trapped inside CIyA nanopore tweezers (FIG. 1B). As a proof of concept, Abl kinase was investigated, of which the active site and allosteric site inhibitors are available. After blocking the active site of the Abl kinase domain with a potent active site inhibitor dasatinib (Tokarski, J. S. et al. Cancer Res 2006 66:5790-5797), additional binding of an allosteric site inhibitor GNF-2 (Zhang, J. et al. Nature 2010 463:501-506) was observed with a distinct current signal, demonstrating the feasibility of the nanopore-based approach for the identification of kinase allosteric inhibitors. Further, the quantification of unknown parameters, such as the kinase conformation transition kinetics in the presence of GNF-2, provides new insight into the kinase inhibition mechanism and shows the versatility of the nanopore assay.

Results

Characterization of Inhibitors Binding to Abl Kinase Domain

In this study, a wildtype variant of the Abl kinase domain referred as _{N4pos}Abl was used. The N-terminal positively charged peptide tag, N4pos (sequence: KRKKSGG, SEQ ID NO:4), did not significantly alter the catalytic kinetics of Abl kinase but helped extend the kinase residence time within the CIyA nanopore (Li, F., et al. Nat Commun 2022 13:3541). First characterized was the binding signal of some reported Abl kinase inhibitors (one myristate site inhibitor, GNF-2 (Zhang, J. et al. Nature 2010 463:501-506), and two ATP site inhibitors, dasatinib (Tokarski, J. S. et al. Cancer Res 2006 66:5790-5797) and imatinib (Schindler, T. et al. Science 2000 289:1938-1942)) individually with the _{N4pos}Abl/CIyA system (FIG. 1A).

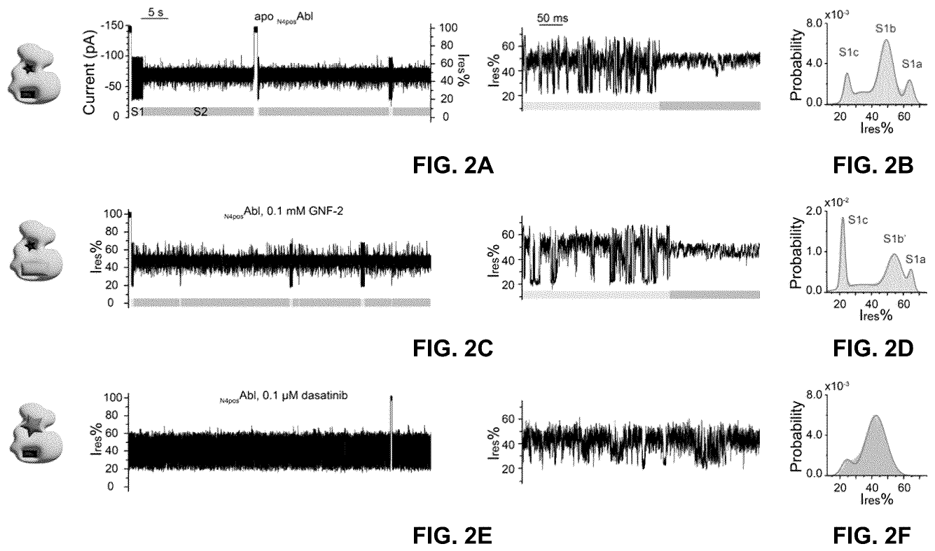

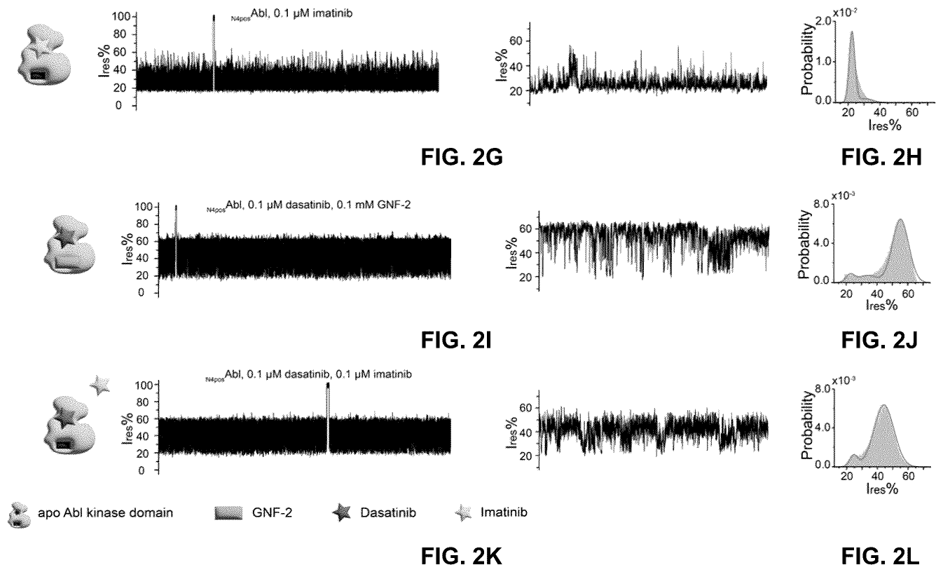

_{N4pos}Abl was added to the cis side of a CIyA nanopore and promoted to enter the nanopore at −80 mV applied voltage bias, resulting in a characteristic current blockage (FIG. 1B-1C). Relative residual current (Iᵣₑₛ%) was calculated from blocked pore current (I_{B}) and open pore current (I_{O}) with Iᵣₑₛ%=100%*I_{B}/I_{O} (FIG. 1C). Two major current states, S1 and S2, were observed with apo _{N4pos}Abl (FIG. 2A) and were to be the lobe-closed and -open conformations respectively (Li, F., et al. Nat Commun 2022 13:3541). The S2 state showed Iᵣₑₛ% ranging from 38-58%. The less populated S1 state, showing Iᵣₑₛ% ranging from 19-69%, consisted of three distinct sub-states (S1a, S1b and S1c) as indicated by the three peaks in the current histogram (FIG. 2B).

When GNF-2 was added to the _{N4pos}Abl/CIyA system, the current signal still showed the alternating S1/S2 pattern, however the S1b state (renamed as S1b′) was shifted by a 5% increase in the Iᵣₑₛ% (S1b: Iᵣₑₛ%=48.4±1.3%, S1b′: Iᵣₑₛ%=53.1±1.7%) (FIG. 2A-2D). Interestingly, GNF-2 binding also affected S1/S2 transition (lobe motions) kinetics. With 0.1 mM GNF-2, the rate constant for lobe opening (k_{S1→S2}) increased by ˜6 folds from 1.86±0.24 s⁻¹ (N=3, n=295; N indicates pore number, n indicates total event number) to 11.23±3.48 s⁻¹ (N=2, n=594) while the rate constant for lobe closing (k_{S2→S1}) didn't change much from 0.09±0.02 s⁻¹ (N=3, n=305) to 0.11±0.01 s⁻¹ (N=2, n=647). Previous studies showed that GNF-2 inhibited kinase activity by allosterically decreasing peptide substrate binding affinity (Choi, Y. et al. J Biol Chem 2009 284:29005-29014). But the detailed mechanisms on how GNF-2 regulate the peptide substrate interactions remains unclear. The current data provided a new clue that GNF-2 might reduce the peptide substrate binding affinity through increasing the lobe open rate.

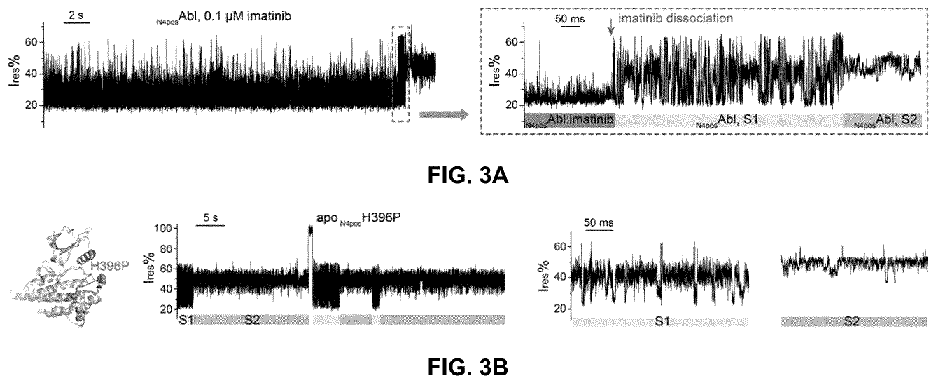

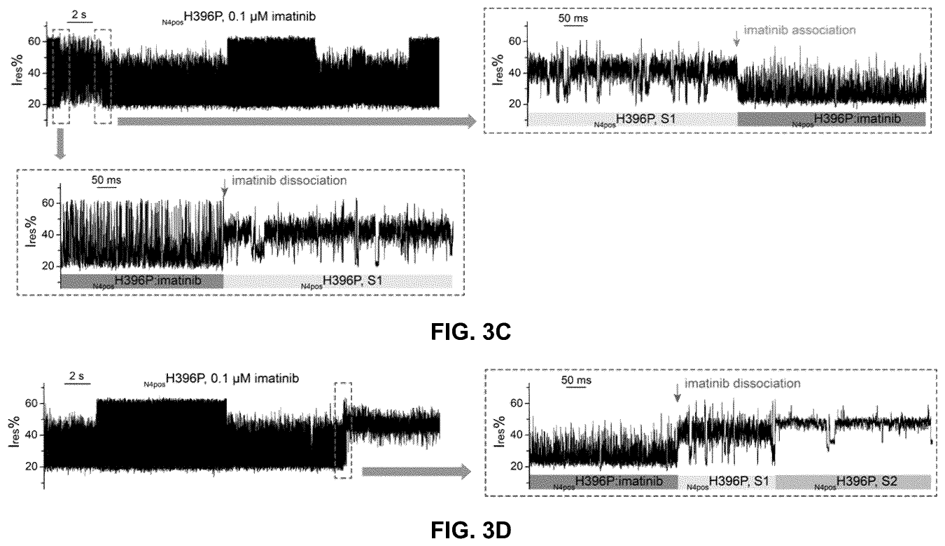

The addition of dasatinib or imatinib depleted the S1/S2 transitions (lobe motions) (FIG. 2E, 2G), which may be due to the observation that dasatinib and imatinib bind to the cleft between the two lobes (FIG. 1A). By binding between the two lobes at the ATP site, dasatinib and imatinib stabilized the lobes in a closed conformation. The dasatinib binding mainly stabilized the current signal at Iᵣₑₛ% of 44.4±1.2% with minor fluctuations to Iᵣₑₛ% of 24.6±0.2% (FIG. 2E-2F). The imatinib binding mainly stabilized the current signal at Iᵣₑₛ% of 23.1±0.6% (FIG. 2G-2H). Due to the fast escape rate of _{N4pos}Abl from the nanopore (1/T_{trapping}=4.8×10⁻² s⁻¹) and the high binding affinity and slow dissociation rate (k_{off}) of dasatinib (K_{D}=0.03±0.01 nM, k_{off}=5.7×10⁻⁴ s⁻¹)³⁸ and imatinib (K_{D}=4±1 nM, k_{off}=7.4×10⁻⁴ s⁻¹) (Hoemberger, M., et al. Proc Natl Acad Sci USA 2020 117:19221-19227; Liang, W. et al. Anal Chem 2014 86:9860-9865) from the Abl, no dissociation events were captured for dasatinib, only one dissociation event was captured for imatinib (FIG. 3A). When testing on a clinically reported drug-resistant Abl mutant, H396P, with reduced imatinib binding affinity (K_{D}=43±3 nM) (Xie, T., et al. Science 2020 370), both imatinib dissociation and association events were captured (FIG. 3B-3D). Together, these results suggested that the inhibitors binding can be well-reported in the ionic current signal and distinguished by the nanopore system.

Detection of GNF-2 in the Presence of Dasatinib

To test the robustness of this nanopore system, we probed the effects of dual inhibitor binding on Abl, with a myristate site inhibitor GNF-2 and an ATP site inhibitor dasatinib. The slow dissociation rate of dasatinib from Abl renders it an active site blocker. Any other compounds that is still capable of interacting with the Abl:dasatinib complex would occupy an allosteric site (non-ATP site). The same rationale has also been used in previous NMR studies for Abl allosteric inhibitor detection (Jahnke, W. et al. J Am Chem Soc 2010 132:7043-7048).

In the nanopore assay, the addition of GNF-2 to the _{N4pos}Abl:dasatinib complex induced a distinct current signal, mainly stabilizing the current at Iᵣₑₛ% of 53.7±1.3% with frequent fluctuations to Iᵣₑₛ% of 23.4±0.4% (FIG. 2I-2J). In addition, the event trapping time was increased by ˜3 folds from 12.71±1.86 s (N=4, n=440) to 41.52±12.49 s (N=4, n=384). As a control, imatinib was tested in the presence of dasatinib where new current signal was observed (FIG. 2K-2L), reinforcing that simultaneous binding of imatinib and dasatinib is unlikely as their binding sites overlap extensively (FIG. 1A). These results showed that the Abl kinase domain is capable of dual binding at distinct inhibitor binding pockets. This perturbation of the _{N4pos}Abl:dasatinib signal by an allosteric site binder is a proof of principle experiment that can be extended to screen for other novel Abl allosteric binders.

### CONCLUSION

With the tests on known active site inhibitors (dasatinib and imatinib) and a myristate site inhibitor (GNF-2), this example demonstrated that the label-free nanopore system, employing an ionic current readout, can identify allosteric compounds with the ATP site blocked with active site inhibitors. There are several advantages of this assay system for allosteric inhibitors detection: 1) requires no fluorescent labels or other probes on the Abl kinase; 2) requires a small amount of Abl kinase (˜picomoles of Abl in a nanomolar concentration); 3) allows for direct measurement of dissociation constants (K_{D}) for the compounds (Li, F., et al. Nat Commun 2022 13:3541); 4) amenable to high-throughput screening; 5) requires no prior knowledge of allosteric sites. This method can also be used to detect compounds that bind to unidentified allosteric sites of Abl kinase, which can potentially help the discovery of novel druggable allosteric sites, design new allosteric inhibitors and novel PROTACs. For the same reason, this method has the potential to be easily adopted to other kinases and drug targets with available active site inhibitors and discover their allosteric compounds.

Methods

Mutagenesis, Expression, and Purification of Abl Kinase

Abl Mutant Plasmids (_{N4pos}Abl and _{N4pos}H396P) were Generated Via PCR with Mutagenesis Primers Listed Below: (N4pos_Forward)

AAACGTAAGAAAAGCGGAGGTTCCCCCAACTACGACAAGTGGGAG (SEQ ID NO:5), (N4pos_reverse) ACCTCCGCTTTTCTTACGTTTTGCATTGGATTGGAAGTACAGG (SEQ ID NO:6); (H396P_forward) CCGGCTGGAGCCAAGTTCCCCATCAAATG (SEQ ID NO:7), (H396P_reverse) CTTGGCTCCAGCCGGGGCTGTGTAGGTGTC (SEQ ID NO:8). The plasmids were verified by Sanger sequencing. The Abl kinase containing plasmid pET_His10 TEV_Abl1 kinase domain (residue 229-512) (Addgene plasmid #79727) and the YopH phosphatase containing plasmid pET_YopH (residues 164-468) (Addgene plasmid #79749) were gifts from John Chodera & Nicholas Levinson & Markus Seeliger, which were co-transformed to BL21(DE3) pLysS competent cells under ampicillin and kanamycin resistance for Abl kinase expression. Co-expression with the YopH phosphatase is to prevent potential autophosphorylation of Abl kinase. Cells were grown in 2xYT media at 37° C., shaking at 200 rpm until OD₆₀₀ₙₘ reached 0.8-1. Cells were then induced by adding isopropyl β-D-1-thiogalactopyranoside (IPTG) to a final concentration of 0.25 mM and incubated at 16° C. for around 16 hours. The cells were harvested by centrifugation at 4,000 g for 30 min at 4° C. and resuspended in lysis buffer (50 mM Tris-HCl, 150 mM NaCl, 5% glycerol, pH 8) and lysed via sonicator (Misonix). The lysate was pelleted by centrifugation at 20 000 g for 25 min at 4° C., and the supernatant was applied to Ni-NTA affinity chromatography for purification. Ten times of column volume of wash buffer containing 50 mM Tris-HCl, pH 8, 500 mM NaCl, 75 mM imidazole, 5% glycerol was used to wash off impurities. Five times of column volume of buffer containing 50 mM Tris-HCl, pH 8, 500 mM NaCl, 200 mM imidazole, 5% glycerol was used to elute the Abl kinase. TEV protease was added to the elute from Ni-NTA purification with a mole ratio of 1:15 (TEV:kinase) to remove the His-tag while dialyzed for 16 hours at 4° C. in dialysis buffer (50 mM Tris-HCl, 100 mM NaCl, 5% glycerol, 1 mM DTT, pH 8). The dialyzed solution was then applied to an anion exchange column to remove TEV and phosphatase. Ten times of column volume of wash buffer containing 50 mM Tris-HCl, pH 8, 100 mM NaCl, 5% glycerol, 1 mM DTT was used to wash off impurities. Five times of column volume of elution buffer containing 50 mM Tris-HCl, pH 8, 200 mM NaCl, 5% glycerol, 1 mM DTT was used to elute His-tag removed Abl kinase. The elute from anion exchange purification was concentrated by centricon with 10 000 Da cutoff and loaded to a size-exclusion column (HWSSS, GE Healthcare Life Sciences) to remove potential aggregates with running buffer (50 mM Tris-HCl, 150 mM NaCl, 5% glycerol, 1 mM DTT, pH 8). The purity of Abl proteins was examined by sodium-dodecyl sulfate polyacrylamide gel electrophoresis (SDS-PAGE).

Kinase Activity Assay

The activities of Abl proteins were measured with a pyruvate kinase-lactate dehydrogenase detection system, where the consumption of ATP was correlated to the oxidation of NADH (Barker, S. C. et al. Biochemistry 1995 34:14843-14851). Reactions were performed with 10 mM MgCl₂, 100 mM Tris-HCl (pH 8.0), 2.2 mM ATP, 1 mM phosphoenolpyruvate, 0.6 mg/mL NADH, 75 U/mL pyruvate kinase, 105 U/mL lactate dehydrogenase, 30 nM kinase and 0.5 mM kinase substrate peptide Abltide (sequence: KKGEAIYAAPFA (SEQ ID NO:9); LifeTein). Absorbance of NADH at 340 nm was monitored with a microtiter plate spectrophotometer (BioTek Synergy 2) at 30° C. for 30 min. The background kinase activity was determined in a reaction mix without the Abltide and subtracted from the experiments with the Abltide. To obtain the enzyme kinetics, initial phosphorylation rates at different Abltide concentration (50-800 μM) were measured. Plots of initial rate as a function of Abltide concentration were fit by nonlinear regression to obtain the Michaelis-Menten kinetic parameters.

Preparation of CIyA Nanopore

The CIyA nanopore used in this study is a mutant CIyA, known as CIyA-AS (Soskine, M., et al. J Am Chem Soc 2013 135:13456-13463). The CIyA-AS containing plasmid pET3a-his5-CIyA-AS was transformed into BL21(DE3) E. coli competent cells and grown in LB media at 37° C. until OD₆₀₀ reached ˜0.6, followed by adding 0.5 mM IPTG for induction and incubated at 16° C. for ˜16 hours with shaking at 200 rpm. The cells were harvested by centrifugation at 4 000 g for 30 min at 4° C. and resuspended in lysis buffer (50 mM Tris-HCl, 150 mM NaCl, 5% glycerol, pH 8) and lysed via sonicator (Misonix). The cells were pelleted by centrifugation at 20,000 g for 25 min at 4° C. The supernatant was then purified with a gravity Ni-NTA affinity column equilibrated with buffer containing 150 mM NaCl, 20 mM imidazole, 50 mM Tris-HCl, pH 8. Wash buffer (150 mM NaCl, 100 mM imidazole, 50 mM Tris-HCl, pH 8) was then applied to the column. The CIyA-AS was eluted with buffer containing 150 mM NaCl, 200 mM imidazole, 50 mM Tris-HCl, pH 8. The elution from the Ni-NTA purification was further applied to a size-exclusion column (HW5SS, GE Healthcare Life Sciences) with running buffer containing 150 mM NaCl, 50 mM Tris-HCl, pH 8. The protein purity was assessed by SDS-PAGE. Proteins were aliquoted then flash frozen with liquid nitrogen and stored at −80° C. for future use. The CIyA-AS monomer was assembled into oligomeric nanopores by incubating with 1% DDM at room temperature for 30 min, then used for current recording experiments.

Single-Channel Recording and Data Analysis

The single-channel recordings were performed with homemade chips. The chip consisted of two chambers (cis and trans) that are separated by a polytetrafluoroethylene film (thickness 25 μm, Goodfellow) with an aperture of around 100 μm in diameter in the film center. To form a lipid bilayer, we first applied 2 μl of 10% hexadecane (v/v in pentane) to both sides of the aperture and then filled both chambers with 300 μl buffer (150 mM NaCl, 100 mM Tris-HCl, pH 7.5), followed by adding 15 μl of 10 mg/ml 1,2-diphytanoyl-sn-glycero-3-phosphocholine (DPhPC, Avanti Polar Lipids) in pentane to both chambers. A DPhPC planar lipid bilayer was formed by pipetting the solution up and down several times. CIyA nanopores were added to the cis chamber (grounded) and then spontaneously inserted into the bilayer, allowing ions to flow through. pClamp 10.7 was used for data recording. After applying a negative voltage bias across the bilayer, the current generated by the ions flow through the nanopore was monitored in voltage-clamp mode by an integrated patch clamp amplifier Axopatch 200B (Molecular Devices). The current signal was acquired by an analog-to-digital converter Digidata 1440A (Molecular Devices) at a sampling rate of 50 kHz after processing with a 4-pole lowpass Bessel filter at 2 kHz. Abl kinase (˜100 nM) was added to the cis chamber and was promoted to enter the nanopore by the applied negative voltage bias. All tested inhibitors (MedChemExpress) were also added to the cis chamber.

Clampfit 11.1 and Igor Pro 9.0 were used for data analysis. Dwell times (T) of trapping events and current states were determined by single-channel search in Clampfit 11.1. All dwell times from one nanopore experiment were binned and fitted to a single-exponential function to derive the T. The average dwell time and the standard deviation were determined from at least two independent nanopore measurements. State transition rate constants were calculated from state dwell times. For example, the transition rate constant from S1 to S2 (k_{S1→S2}) was determined from S1 state dwell time (T_{S1}) with k_{S1→S2}=1/T_{S1}. The average state transition rate constant and the standard deviation were determined from at least two independent nanopore measurements. Of note in calculating T_{S1} and T_{S2}, the S1 or S2 states at the beginning and those at the end of a trapping event were discarded as their true dwell times were interrupted by kinase entering and exiting the CIyA nanopore. Relative residual current (Iᵣₑₛ%) was calculated from blocked pore current (I_{B}) and open pore current (I_{O}) with Iᵣₑₛ%=100%×I_{B}/I_{O}. The Iᵣₑₛ% value of the peak center in the histograms were derived from Gaussian multipeak fitting using Igor Pro 9.0. The average Iᵣₑₛ% value of peak centers and the standard deviation were determined from three independent nanopore measurements.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A nanopore tweezer system comprising:
(i) a fluid-filled compartment separated by a membrane into a first chamber and a second chamber, wherein the fluid is an ionic solution;
(ii) a CIyA nanopore disposed in the membrane with a cis pore lumen of about 7 nm and a trans pore lumen of about 3 nm;
(iii) a protein kinase in the lumen of the CIyA nanopore; and
(iv) electrodes configured for generating an electrical potential difference across the membrane to facilitate ionic flow through the CIyA nanopore from the first chamber to the second chamber,
wherein the protein kinase has an active site that is blocked with an inhibitor.

2. The nanopore tweezer composition of claim 1, wherein protein kinase has a molecular weight in the range of 15-70 kDa.

3. The nanopore tweezer composition of claim 1, wherein the protein kinase is tyrosine kinase, and wherein the inhibitor is a tyrosine kinase inhibitor.

4. The nanopore tweezer composition of claim 2, wherein the protein kinase is selected from the group consisting of Abl1, ACK, ALK, ARG (Abl2), Axl, BRK, BTK, CTK/MATK, EGFR, Eph family, EphA1, EphA2, EphA3 (HEK), EphB2, EphB4 (HTK), FAK (PTK2), FER, FES, FGFR1, FGFR2, FGFR3, FGFR4, FGR, FLT1 (VEGFR1), FLT3, FLT4 (VEGFR3), FMS (CSF1R), Fyn, HER2 (ErbB2), HER3 (ErbB3), HER4 (ErbB4), IGF1R, INSR, ITK, Jak1, Jak2, Jak3, KDR (FLK1, VEGFR2), Kit, LCK, LTK, LYN, Mer, Met, MusK, PDGFRα, PDGFRβ, PYK2, Ret, RON, ROR2, ROS, SRC, Syk, Tie2 (TEK), TrkA (NTRK1), TrkB (NTRK2), TrkC (NTRK3), Tyk2, TYRO3 (SKY), Yes, and Zap-70, or a mutant variant thereof.

5. The nanopore tweezer composition of claim 1, wherein the inhibitor is an ATP-competitive inhibitor.

6. The nanopore tweezer composition of claim 4, wherein the inhibitor is selected from the group consisting of dasatinib. imatinib, nilotinib, AIM-100, KRCA-0008, dorsomorphin, A-443654, TAE684, DMH-1, ML347, AZ12601011, AZD0156, KU-60019, Elimusertib, Gartisertib, VE-821, AK-01, Cabozantinib, @ARK1 Inhibitor, Dorsomorphin, AZ304, DP-4978, LXH254, B12536, SF2523, Cpd 4f, XMU-MP-3, 2OH-BNPP1, BAY-524, AT7519, BMS-265246, SU9516, AT7519, Dinaciclib, Prexasertib, Prexasertib dihydrochloride, Prexasertib dimesylate, IC261, Hematein, Tpl2 Kinase Inhibitor 1, TC-DAPK 6, HS38, BAY-8400, MBM-55, NH125, Erlotinib Hydrochloride, Lapatinib, ALW-II-41-27, NVP-BHG712, AWL-II-38.3, JI-101, Tesevatinib, XMD8-92, AX-15836, BAY885, Conteltinib, Defactinib, E260, Herbimycin A, ON123300, ENMD-2076, Infigratinib, ENMD-2076, Saracatinib, Linifanib, Cediranib, Linifanib, GW2580, TG 100572 Hydrochloride, PP2, RGB-286638, Indirubin-3′-monoxime, Varlitinib, Epertinib hydrochloride, Sapitinib, PF-06260933, Protein kinase inhibitors 1 hydrochloride, AZD-3463, Resveratrol, PS-1145, IMD-0560, TBK1/IKKε-IN-2, TBK1/IKKε-IN-5, ILK-IN-3, Ceritinib, IRAK inhibitor 2, Takinib, PF-06426779, HS271, BMS-509744, AZD-1480, SAR-20347, Ritlecitinib, AS601245, WHI-P258, Tyrphostin AG1433, Sorafenib, GA-017, S116836, SM1-71, Pim1/AKK1-IN-1, LRRK2-IN-1, GSK2578215A, R406, MKI-1, Trametinib, JNJ-47117096 hydrochloride, MELK-8a hydrochloride, UNC569, LDC1267, Tivantinib, Gossypetin, HRX-0215, Hesperadin, Rapamycin, AZ-23, ZINC05007751, JH 295, rac-CCT 250863, Talmapimod, RWJ-67657, LY-2584702 tosylate salt, FRAX597, FRAX486, GNE 2861, FRAX1036, Seralutinib, ISRIB (trans-isomer), GSK2656157, TCS PIM-1 1, SGI-1776, SEL24-B489, SGI-1776, SEL24-B489, 5-lodotubercidin, Staurosporine, Ruboxistaurin, Enzastaurin, Staurosporine, Mallotoxin, Spheciosterol sulfate A, Staurosporine, Sotrastaurin, PKR-IN-C16, PKR-IN-C51, Wortmannin, Conteltinib, TAK-632, ON123300, RKI 1447 dihydrochloride, AMG-208, BMS-777607, Lorlatinib, BRD7389, BIX 02565, BI-D1870, GSK 650394, PP121, Syk Inhibitor II, Piceatannol, Midostaurin, EW-7195, LY3200882, GW788388, S116836, Cabozantinib, Altiratinib, Entrectinib, Gandotinib, BMS-777607, WNK463, SU6656, PD173955, and RDN009.

7. The nanopore tweezer composition of claim 1, wherein the membrane preparation comprises a planar lipid bilayer.

8. The nanopore tweezer composition of claim 6, wherein the membrane preparation comprises a micelle, a bacterium, or a eukaryotic cell.

9. The nanopore tweezer composition of claim 1, wherein the protein kinase has been modified with an N-terminal positively charged peptide tag to extend the kinase residence time.

10. The nanopore tweezer composition of claim 8, wherein the N-terminal positively charged peptide tag comprises the amino acid sequence KRSGGKK (SEQ ID NO:10), KRKKSKGG (SEQ ID NO:11), or KRKKSGG (SEQ ID NO:12).

11. The nanopore tweezer composition of claim 1, wherein the CIyA pore comprises 10 to 14 subunits, wherein each subunit of the CIyA nanopore has the amino acid sequence SEQ ID NO:1, or a variant thereof having at least 80% sequence identity to SEQ ID NO:1.

12. The nanopore tweezer composition of claim 10, wherein each subunit of the CIyA nanopore has at least one C87S, C87A, C285S or C285S substitution.

13. The nanopore tweezer composition of claim 10, wherein each subunit of the CIyA nanopore has at least one L99, E103, F166, and K294 substitution.

14. The nanopore tweezer composition of claim 10, wherein each subunit of the CIyA nanopore has the amino acid sequence SEQ ID NO:2 or SEQ ID NO:3.

15. A method for screening for an allosteric binder to a protein kinase, comprising:
(a) providing the nanopore tweezer system of claim 1,
(b) assessing a gating pattern of the nanopore,
(c) adding a candidate agent to the first chamber and/or the second chamber;
(d) assessing the gating pattern for a change after step (c); and
(e) comparing gating patterns from step (b) to step (d), wherein a change in the gating pattern is an indication that the candidate agent binds the peptide kinase at an allosteric site.


SEQUENCE LISTING (12 sequences)

SEQ ID NO: 1 - protein, 312 aa, synthetic construct
MTGIFAEQTVEVVKSAIETADGALDLYNKYLDQVIPWKTFDETIKELSRFKQEYSQEASVLVGDIKVLLMDSQDKYFEATQTVYEWAGVVTQLLSAYIQLFDGYNEKKASAQKDILIRILDDGVKKLNEAQKSLLTSSQSFNNASGKLLALDSQLTNDFSEKSSYYQSQVDRIRKEAYAGAAAGIVAGPFGLIISYSIAAGVVEGKLIPELNNRLKTVQNFFTSLSATVKQANKDIDAAKLKLATEIAAIGEIKTETETTRFYVDYDDLMLSLLKGAAKKMINTSNEYQQRHGRKTLFEVPDVGSSYHHHHH

SEQ ID NO: 2 - protein, 304 aa, synthetic construct
MTGIFAEQTVEVVKSAIETADGALDLYNKYLDQVIPWKTFDETIKELSRFKQEYSQEASVLVGDIKVLLMIDSQDKYFEATQTVYEWCGVVTQLLSAYIQLFDGYNEKKASAQKDILIRILDDGVKKLNEAQKSLLTSSQSFNNASGKLLALDSQLTNDFSEKSSYYQSQVDRIRKEAYAGAAAGIVAGPFGLIISYSIAAGVIEGKLIPELNNRLKTVQNFFTSLSATVKQANKDIDAAKLKLATEIAAIGEIKTETETTRFYVDYDDLMLSLLKGAAKKMINTSNEYQQRHGRKTLFEVPDV

SEQ ID NO: 3 - protein, 303 aa, synthetic construct
MTGIFAEQTVEVVKSAIETADGALDLYNKYLDQVIPWKTFDETIKELSRFKQEYSQEASVLVGDIKVLLMDSQDKYFEATQTVYEWAGVVTQLLSAYIQLEDGYNEKKASAQKDILIRILDDGVKKLNEAQKSLLTSSQSFNNASGKLLALDSQLTNDFSEKSSYYQSQVDRIRKEAYAGAAAGIVAGPFGLIISYSIAAGVVEGKLIPELNNRLKTVQNFFTSLSATVKQANKDIDAAKLKLATEIAAIGEIKTETETTRFYVDYDDLMLSLLKGAAKKMINTSNEYQQRHGRKTLFEVPDV

SEQ ID NO: 4 - protein, 7 aa, synthetic construct
KRKKSGG

SEQ ID NO: 5 - DNA, 45 bp, synthetic construct
  source 1..45: mol_type = other DNA
aaacgtaagaaaagcggaggttcccccaactacgacaagtgggag

SEQ ID NO: 6 - DNA, 43 bp, synthetic construct
  source 1..43: mol_type = other DNA
acctccgcttttcttacgttttgcattggattggaagtacagg

SEQ ID NO: 7 - DNA, 29 bp, synthetic construct
  source 1..29: mol_type = other DNA
ccggctggagccaagttccccatcaaatg

SEQ ID NO: 8 - DNA, 30 bp, synthetic construct
  source 1..30: mol_type = other DNA
cttggctccagccggggctgtgtaggtgtc

SEQ ID NO: 9 - protein, 12 aa, synthetic construct
KKGEAIYAAPFA

SEQ ID NO: 10 - protein, 7 aa, synthetic construct
KRSGGKK

SEQ ID NO: 11 - protein, 8 aa, synthetic construct
KRKKSKGG

SEQ ID NO: 12 - protein, 7 aa, synthetic construct
KRKKSGG